(19) United States
(12) Patent Application Publication
(10) Pub. No.: US 2026/0263815 A1
(43) Pub. Date: Sep. 10, 2026

(54) Mode Adjustment Arrangement

(71) Applicant: BIOTRONIK SE & Co. KG, Berlin (DE)

(72) Inventors: Frank BECKER, Berlin (DE); Volker LANG, Berlin (DE); Sebastian SCHULZE-LUCKOW, Berlin (DE); Thomas DOERR, Berlin (DE)

(73) Assignee: BIOTRONIK SE & Co. KG, Berlin (DE)

(21) Appl. No.: 19/166,316

(22) PCT Filed: Mar. 4, 2024
(86) PCT No.: PCT/EP2024/055573

§ 371 Date: Sep. 17, 2025

(30) Foreign Application Priority Data
Mar. 21, 2023 (EP) 23163058.3

(51) Int. Cl. A61N 1/372; A61N 1/39

(52) U.S. Cl. CPC A61N 1/37211; A61N 1/39622

(57) ABSTRACT

An arrangement including a first device and a second device, wherein the first device and the second device can be operatively coupled with each other to transfer data at least from the second device to the first device, wherein the first device is an implanted medical device for cardiac therapy of a patient, wherein the first device is designed and arranged such to activate an operational mode of the first device in dependence on the data transferred by the second device. The second device is not a programming device serving for programming a medical device. The activated operational mode is chosen from the group consisting of: a normal mode in which the first device is operated with standard functionalities, and at least one second mode, wherein the second mode is chosen from at least one of: a protected mode, an increased stress mode, a fitness mode and a mobility mode.

### CROSS-REFERENCE TO RELATED APPLICATIONS

This application is the United States National Phase under 35 U.S.C. § 371 of PCT International Patent Application No. PCT/EP2024/055573, filed on Mar. 4, 2024, which claims the benefit of European Patent Application No. 23163058.3, filed on Mar. 21, 2023, the disclosures of which are hereby incorporated by reference herein in their entireties.

### TECHNICAL FIELD

The present invention relates to an arrangement according to the preamble of claim 1 and to a method for operating a first device and a second device according to the preamble of claim 14.

### BACKGROUND

Prior art in the field of implantable medical devices teaches many possibilities of changing or activating an operative state of the implantable medical devices. To give some examples, WO 2017/189558 A1 and US 2021/0187313 A1 describe an implantable cardioverter-defibrillator (ICD) that can switch its operative mode from a sensing without pacing mode to a temporary pacing mode in response to the detection of an asystole.

WO 2017/189676 A1 describes an implantable medical device that can switch its operational mode from a normal pacing mode to a post-shock pacing mode upon detection of the delivery of an anti-tachyarrhythmia shock by a second medical device. The detection of the anti-tachyarrhythmia shock can be accomplished by detecting the shock pulse itself or by detecting an asystole by the implantable medical device.

US 2017/0296835 A1 describes an implantable medical device that can be switched into an exposure mode of operation by an automatic detection of an exposure to magnetic disturbance. The concrete functionality employed by the implantable medical device in the exposure mode of operation can depend on further parameters.

US 2020/0101298 A1 describes an implantable medical device that automatically determines at least some of the parameters of an exposure operating mode based on stored information regarding sensed physiological events or therapy provided over a predeterminable period of time. Thus, this implantable medical device is capable of configuring itself to operate in accordance with the automatically determined parameters.

Furthermore, it is well known to use an external programming device for programming an implanted medical device and to switch the operational mode of this implanted medical device.

If two or more implantable medical devices are implanted within the same patient, they may negatively influence each other. Furthermore, different implanted medical devices may have different functionalities that can only be used by the implantable medical device that features the respective functionality.

The present disclosure is directed toward overcoming one or more of the above-mentioned problems, though not necessarily limited to embodiments that do.

### SUMMARY

It is an object of the present invention to overcome the drawbacks of prior art, in particular to reduce the risk of a negative influence of different implanted medical devices with respect to each other and/or to increase the functionalities of an implanted medical device.

This object is achieved with an arrangement having the claim elements of claim 1. Such an arrangement comprises a first device and a second device. The first device and the second device can be operatively coupled with each other to transfer data at least from the second device to the first device (optionally also from the first device to the second device). The first device is an implanted medical device for cardiac therapy of a patient. The first device is able to activate an operational mode of the first device in dependence on the data transferred by the second device. Expressed in other words, the first device is designed and arranged such to activate the operational mode in dependence on the data transferred by the second device.

According to the claimed invention, the second device is different from a programming device, i.e., it is explicitly not a programming device that typically serves for programming a medical device. In addition, the activated operational mode is chosen from the group consisting of a plurality of different operational modes that will be explained in the following.

A first possible operational mode is a normal mode in which the first device is operated with standard functionalities. Such a normal mode can also be denoted as standard mode.

The operational mode is additionally chosen from at least one second mode, wherein the second mode is chosen from at least one of:

A protected mode in which the first device is operated without specific safety functions. The omission of specific safety functions can be done since the failure of the first device would be compensated by the second device in the protected mode. Expressed in other words, the second device serves as backup device for the first device and thus allows an operation of the first device in the protected mode.

A further possible operational mode is an increased stress mode that is adapted to an expected increased level of stress of the patient and that features an adapted rate and/or an adapted timing of a pacing to be applied to the patient. Particularly, the increased level of stress of the patient is expected to be accompanied by an increase heart rate and/or increased blood pressure. However, a too increased heart rate is not desirable. Thus, in an embodiment, the first device in an activated stress mode may be configured to adjust the pacing rate only up to a moderate value, e.g., a stress pacing rate threshold.

According to an embodiment, the first device in an activated increased stress mode is configured to perform at least one of:
cardiac pacing using a reduced or increased pacing rate, particularly an increased pacing rate up to a stress pacing rate threshold,
cardiac pacing using an adjusted AV delay,
cardiac pacing using adjusted parameters for detection of ventricular tachycardia (VT)/ventricular fibrillation (VF), like e.g. by applying increased rate limits.

A further possible operational mode is a fitness mode that is adapted to an expected increased level of physical activity of the patient. Also in this fitness mode, the rate and/or timing of a possible pacing for the patient is adapted to the condition of the patient's heart that will be assumed by the heart in case of an increased physical activity of the patient. Particularly, an increased physical activity of the patient is expected be accompanied by an increased heart rate and respiratory rate to meet the physiological demand of the body (oxygen, nutrients) in response to the increased physical activity of the patient. Particularly, a first device may be configured to adjust the pacing rate to achieve an optimal training heart rate. The optimal training heart rate may differ for the actual physical active, e.g., the optimal training heart rate may be different for cardio training than for strength training.

According to an embodiment of the present invention, the first device in an activated fitness mode is configured to perform at least one of:
cardiac pacing using adjusted parameters for rate adjustment (e.g. based on accelerometer data or based on cardiac contractility data derived from intracardiac impedance measurements),
cardiac pacing using a shortened AV delay,
cardiac pacing using adjusted VT/VF detection parameters like increased rate limits.

Another possible operational mode is a mobility mode in which a motion sensor of the first device is deactivated since the patient is expected to move with the help of a transport device and thus in a higher velocity than a human patient without any transport device.

According to an embodiment, a further possible operational mode is a danger mode in which the first device will deliver, during a first predeterminable period of time, cardiac pacing pulses with an increased amplitude and/or an increased rate. Such pacing with an increased amplitude and/or an increased rate is a particularly appropriate post-shock measure for pacing a heart that has been subjected to a defibrillation pulse.

Furthermore, according to an exemplary embodiment, a further possible operational mode is a magnetic resonance imaging mode in which the first device (or at least a pacing functionality of the first device) is deactivated (e.g., for a second predeterminable period of time) and subsequently reactivated after the second predeterminable period of time has passed or upon detection of an absence of the previously present magnetic field. This magnetic resonance imaging mode is a particularly appropriate operational mode if the patient is subjected to a magnetic resonance imaging (MRI) measurement in a hospital or a medical institution or if the patient is subjected to a security check such as a security check at an airport.

Moreover, according to an aspect of the present invention, the first device in an activated mobility mode is configured not to use motion sensor data to adjust the cardiac pacing therapy. Alternatively, the first device is configured to use adjusted motion sensor of which accelerations not caused by patient body movement itself are compensated for adjusting the cardiac pacing therapy.

In contrast to prior art solutions, the first medical device is not dependent on specific sensing functionalities in order to detect a specific physiological state of the patient or an environmental condition the patient is subjected to. Rather, the first medical device is provided with data from the second device and switches its operational mode (i.e., it activates a specific operational mode in particular instead of the previously activated operational mode) in dependence on this data. Thus, there is an active communication at least from the second device to the first device.

Due to this interaction between the first device and the second device, the risk that the first device and the second device negatively influence each other is also significantly reduced. If the second device is known to be device that can negatively influence medical devices, it may thus communicate its presence or its activity or its activity in specific operational mode to the first device by transferring the data comprising respective information to the first device. Thus, this active data transfer from the second device to the first device reduces the risk of an interference between the first device and the second device since the first device can then be operated in an operational mode that is less prone to interference with the second device.

Furthermore, by transferring data from the second device the first device, specific functionalities of the second device can be indirectly used by the first device. To give an example, sensors being present only in the second device, but not in the first device, can be used to detect a specific condition or state of the patient and. Information on this condition or state can then be transferred as part of the data to the first device. The first device is then capable of adjusting its operational mode based on information that the first device alone would not be able to collect. This increases the functional versatility of the first device without requiring any hardware modification of the first device (despite the general capability of the first device of receiving data from the second device).

In an embodiment, the first device is designed and arranged to transfer data to the second device. By doing so, the first device can, e.g., indicate its presence to the second device and thus request the transfer of data from the second device.

In an embodiment, the second device actively looks for a presence of the first device prior to transferring data to the first device. Such an active search for the first device can be performed by emitting search signals and waiting for an according response or, alternatively, for a specific (passive) reflection of an emitted signal.

In an embodiment, the first device actively looks for the second device. Such an active search by the first device can be performed, in general, like in case of an active search for the first device by the second device.

In an embodiment, the first device and the second device actively search for the presence of each other device. This active “double search” reduces the time being necessary for locating the respective other device, but requires a higher amount of energy than in case of an active search performed only by a single device.

The active search can be performed, in an embodiment, according to a fixed or adjustable schedule, e.g., at a specific time of day or according to operative hours of the first and/or the second device. Alternatively, the active search can be done in time intervals that are determined by artificial intelligence.

In an embodiment, the data transfer from the second device to the first device and optionally from the first device to the second device is accomplished in a wireless manner. All standard data transmission protocols or specifications are appropriate for such a wireless data communication. Examples of standard data transmission protocols or specifications are the Medical Device Radiocommunications Service (MICS), the Bluetooth Low Energy (BLE) protocol, the Zigbee specification, the long range wide area network (LoRaWAN) protocol, the wireless personal area network (WPAN) specification, the low-power wide-area network (LPWAN) specification, the wireless local area network (WLAN) specification, the Global System for Mobile Communications (GSM) specification, the Long-Term Evolution (LTE) standard, and the fifth-generation technology standard for broadband cellular networks (5G).

In an embodiment, the first device is an implantable pulse generator (IPG), an implantable leadless pacemaker (iLP), or an implantable cardioverter-defibrillator (ICD), in particular a subcutaneously implantable cardioverter-defibrillator (S-ICD). These devices are particularly appropriate cardiac therapy devices.

In an embodiment, the second device is also an implantable medical device for cardiac therapy, wherein it is implanted to the same patient as the first device. In an embodiment, the second device is an IPG, an iLP, or an ICD, in particular an S-ICD.

In an embodiment, the first device and the second device are devices of the same type, e.g., the first device and the second device are both IPGs, both iLPs, or both ICDs like S-ICDs. In such a constellation, the second device can typically not supplement the first device with additional functionalities. However, it can take over functionalities of the first device that cannot be longer accomplished by the first device, e.g., due to failure or to a low battery capacity. Thus, this constellation is particularly useful near to the end of life of the first device and allows an optimal usage of the battery capacity of the first device.

In an embodiment, the first device and the second device are explicitly not the same type of device, but rather different types of devices. Then, the second device can supplement the functionalities of the first device by providing data to the first device that has been gathered by the second device but could not have been gathered by the first device. Expressed in other words, the first device and the second device form a network or association in which at least data gathered by the second device can be transferred to the first device. In an embodiment, also data gathered by the first device can be transferred to the second device. Then, both devices have access to data on the physiologic state of the patient or on data on environmental conditions of the patient that could not be obtained by the first device and/or the second device alone.

In an embodiment, the second device is a device that is capable of providing location information of the user of the second device. Typically, the user of the second device is the patient carrying the first device in an implanted state. Then, the location information of the patient can be transferred as the data to the first device. Consequently, the first device is able to rely on location data without being itself capable of identifying a location of the patient.

In an embodiment, the second device is a smartphone. Smartphones typically have a GPS or other location sensor and can thus provide very specific information on the concrete position or location of the user of the smart phone, e.g., the patient carrying the first device in an implanted state.

In an embodiment, the first device is an IPG or an iLP and the second device is likewise an IPG or iLP. In particular, the first device and the second device are the same type of cardiac therapy device. Furthermore, the data provided by the second device indicates that the second device has an essentially fully charged battery, wherein the activated operational mode of the first device is the protected mode. “Essentially fully charged” means that the capacity of the battery is reached to an extent of at least 90%, in particular at least 95%, in particular at least 97%, in particular at least 98%, in particular at least 99%, in particular 100%. Due to the essentially fully charged battery of the second device, the second device can take over the functionalities of the first device so that it is not necessary that the first device is operated in a particularly energy-saving operational mode. Rather, the protected mode can be activated in which the operation of the first device is safeguarded by the second device that can take over the functionality of the first device in case that the first device is no longer capable of performing its activities.

In an embodiment, the first device is an IPG or an iLP and the second device is an S-ICD. In this context, the data provided by the second device indicates that the second device has emitted a defibrillation pulse. Because of this data, the activated operational mode of the first device is the danger mode. As explained above, in the danger mode the first device applies a pacing with an increased rate and/or an increased amplitude. Since the stimulation threshold of the patient's heart is increased due to the defibrillation pulse, such an increased rate and/or amplitude of subsequently applied pacing pulses is a particularly physiologic measure for proper stimulation of the patient's heart after having received a defibrillation pulse. It should be noted that the first device does not detect the defibrillation pulse itself. Thus, it is not necessary that the sensing unit of the first device is directly or indirectly capable of sensing such defibrillation pulse. Rather, the second device, i.e., the S-ICD, provides corresponding information with the data transferred to the first device indicating that a defibrillation pulse has been emitted. This data is decisive for the first device to change its operational mode, i.e., to activate the danger mode as operational mode.

In an embodiment, the first device is designed and arranged such to switch the operational mode of the first device back from the danger mode to the normal mode after the first predeterminable period of time has passed. This ensures that the first device is operated in a physiologic sensible manner since an increased stimulation threshold will only persist during a limited period of time after having received a defibrillation pulse. In an embodiment, the first predeterminable period of time is a time lying in a range of from 1 minute to 60 minutes, in particular from 2 minutes to 50 minutes, in particular from 3 minutes to 45 minutes, in particular from 4 minutes to 40 minutes, in particular from 5 minutes to 35 minutes, in particular from 6 minutes to 30 minutes, in particular from 7 minutes to 25 minutes, in particular from 8 minutes to 20 minutes, in particular from 9 minutes to 20 minutes, in particular from 10 minutes to 15 minutes.

In an embodiment, the first device is an IPG or an iLP and the second device is an S-ICD, wherein the data provided by the second device indicates that the patient is subjected to a magnetic field. Then, the activated operational mode of the first device is the magnetic resonance imaging mode. Thus, the first device can be switched into this magnetic resonance imaging mode without being itself capable of detecting that magnetic field. Rather, the second device assists with its magnetic field detection capabilities the functionalities of the first device. Even if the first device is capable of detecting a magnetic field, the second device -due to its subcutaneous implantation site-is typically able to detect a magnetic field earlier than the first device. Thus, the sensitivity threshold of the first device with respect to a detection of a magnetic field is increased without changing the hardware of the first device, but simply by providing the data from the second device to the first device. Consequently, the first device can be switched earlier into the magnetic resonance imaging mode than if it needed to rely on its own magnetic field detection capabilities (if any).

Since a magnetic field can generally have different origins that require a different response of the first device to the detected magnetic field, the first device can, in an embodiment, be controlled in a specific way addressing this issue. In this embodiment, the first device is designed and arranged such to deactivate a pacing function of the first device if the patient is in a lying position. Furthermore, the first device is designed and arranged such to reactivate the pacing function and to switch the operational mode of the first device to a normal mode after having been provided with data from the second device that the patient is no longer subject to the magnetic field. A lying position of the patient indicates that the patient is subject to a magnetic resonance imaging examination that typically takes a longer period. In order not to interfere with this magnetic resonance imaging examination, the first device is deactivated. This deactivation can be done for a second predeterminable period of time or without specific time limitation. If the second device detects that the previously present magnetic field is no longer present, it can send data containing this information to the first device, thus serving for a reactivation of the first device.

Alternatively or additionally, the first device is designed and arranged such to deactivate the pacing function of the first device for the second predeterminable period of time if the patient is in an upright position and to reactivate the pacing function and to switch the operational mode of the first device to the normal mode again after the second predeterminable period of time has passed. An upright position of the patient typically indicates that the patient is not subject to a magnetic resonance imaging examination. Rather, a magnetic field and an upright position of the patient indicates that the patient is subject to a security check, e.g., at an airport. Since such a security check does typically not last longer than a couple of minutes, an automatic reactivation without positive knowledge on an absence of the previously present magnetic field can be typically applied without danger for the first device or the patient.

The second predeterminable period of time is, in an embodiment, a time period lying in a range of from 1 minute to 60 minutes, in particular from 2 minutes to 50 minutes, in particular from 3 minutes to 45 minutes, in particular from 4 minutes to 40 minutes, in particular from 5 minutes to 35 minutes, in particular from 6 minutes to 30 minutes, in particular from 7 minutes to 25 minutes, in particular from 8 minutes to 20 minutes, in particular from 9 minutes to 20 minutes, in particular from 10 minutes to 15 minutes.

In case of an upright position of the patient, the second predeterminable period of time is in particular a time period lying in a range of from 1 minute to 6 minutes, in particular from 2 minutes to 5 minutes, in particular from 3 minutes to 4 minutes.

In an embodiment, the first device and/or the second device comprises a position sensor for determining whether the patient is in a lying position on in an upright position. In an embodiment, such a position sensor is implemented in the second device so that the data transferred from the second device to the first device also comprises position data of the patient. In another embodiment, the position data is provided to the first device from a third device that comprises a position sensor. The data transfer from the third device to the first device can be accomplished in a direct way or in an indirect way, e.g., via the second device.

In an embodiment, the activated operational mode of the first device is chosen in dependence on the location information, if the second device is a device providing location information of the user of the second device. In this context, the normal mode is activated if the location information indicates that the patient is at home. Then, no particular pacing adjustments appear to be necessary. Alternatively or additionally, the increased stress mode is activated if the location information indicates that the patient is at work. Alternatively or additionally, the fitness mode is activated if the location information indicates that the patient is at a place at which the patient typically performs sportive activities. Such a place can be, e.g., a fitness studio or a sports ground. Alternatively or additionally, the mobility mode is activated if the location information indicates that the patient is in a moving transport device, in particular in a moving car. All of the previously explained embodiments take advantage of specific location information that is assigned to discrete sites from which it is known how the patient typically behaves at these sites.

In an embodiment, the mobility mode additionally features the functionalities of the increased stress mode. Then, the mobility mode takes into account a potentially increased stress level of the patient assuming that the patient is driving a car.

In an embodiment, the second device is a ventricular assist device (VAD), in particular a left ventricular assist device (LVAD). The data transferred from the VAD to the first device comprises measuring data on a physiologic parameter of the patient, such as the blood pressure of the patient. The data furthermore comprises device data such as a flow rate and a viscosity of the transported liquid. The viscosity can be determined from a power uptake of the VAD. The first device is, in this embodiment, operated in a normal mode if the VAD is not active.

In an embodiment, the second device is a device for deep brain stimulation (DBS), also referred to as brain pacemaker. Such a DBS can be applied, e.g., for the treatment of epilepsy. By transferring data from the DBS device to the first device, in particular in case of the first device being an IPG or an ICD, it is possible to include the first device in a closed-loop stimulation (CLS) control loop of a DBS therapy. Then, the first device can deliver to the patient's heart a pacing with a rate and intensity that is specifically adjusted to the DBS therapy, e.g. the timing of the cardiac pacemaker therapy can be adjusted according to an epilepsy therapy performed by the DBS device. Alternatively or in addition, the DBS therapy is specifically adjusted to the cardiac pacing therapy of the first device. Moreover, according to an embodiment, the DBS data can be used to optimize cardiac rate adaptation therapy performed by the first device. In particular, the DBS data collected during an epilepsy-free episode is used to determine the mental state of a patient, and the cardiac stimulation rate performed by the first device adapted accordingly.

In an independently claimed aspect, the present disclosure relates to an arrangement comprising a first device and a second device, wherein the first device and the second device can be operatively coupled with each other to transfer data at least from the second device to the first device, wherein the first device is an implanted medical device for cardiac therapy of a patient, wherein the first device is designed and arranged such to activate an operational mode of the first device in dependence on the data transferred by the second device (in particular data comprising measuring data on a physiologic parameter of the patient, such as the blood pressure of the patient, and device data such as a flow rate and a viscosity of the transported liquid). According to this aspect of the present disclosure, the second device is not a programming device serving for programming medical device, but a VAD. In addition, the operational mode of the first device that is activated is a VAD mode in which the applied algorithms are more interference-resistant than the algorithms applied in the normal mode.

In an independently claimed aspect, the present disclosure relates to an arrangement comprising a first device and a second device, wherein the first device and the second device can be operatively coupled with each other to transfer data at least from the second device to the first device, wherein the first device is an implanted DBS device for deep brain stimulation of the patient, wherein the first device is designed and arranged such to activate an operational mode of the first device in dependence on the data transferred by the second device. According to this aspect of the present invention, the second device is not a programming device serving for programming medical device, but a device for cardiac therapy of the patient, in particular an IPG or an ICD. In addition, the operational mode of the first device that is activated is a synchronized stimulation mode in which the DBS therapy is synchronized with the cardiac rhythm of the patient. Such a synchronization of the cardiac pacing with the DBS therapy results in a more efficient DBS therapy.

An independently claimed aspect of the present disclosure relates to an arrangement comprising a first device and a second device, wherein the first device and the second device can be operatively coupled with each other to recognize the presence of the respective other device. The first device is an implanted medical device for cardiac therapy of a patient. The second device is a coupling device (such as a sensor) being located in, at, or under a bed of the patient. If the first device and the second device couple with each other (irrespective of any specific data transfer between the devices), this indicates that the patient lies in his bed. Consequently, a night mode is activated upon such coupling between the first device and the second device. If, however, the coupling between the first device and the second device is interrupted or not established at all, this indicates that the patient is not located in his bed so that the first device is operated in a day mode. In the night mode, a stand-up support for elderly patients can be activated. Furthermore, a rate decrease can be activated in the night mode. Furthermore, patient monitoring by medical staff can be performed at longer intervals since a significant physiologic change during the patient's stay in the bad is not to be expected. In contrast, the intervals of a patient monitoring are decreased in the day mode since daily activities of the patient may require an adaptation of the patient's heart to different environmental conditions. This might not be easily accomplished by the patient's heart depending on the health status of the patient.

In an embodiment of this aspect, the first device and/or the second device (regularly or at least in intervals) sends out data to the respective other device that is to be received by the other device. In another embodiment, only signals for passively identifying the respective other device are emitted by the first device and/or the second device.

An independently claimed aspect of the present disclosure relates to an arrangement comprising a first device and a second device, wherein the first device and the second device can be operatively coupled with each other to transfer data at least from the second device to the first device, wherein the first device is an implanted medical device for cardiac therapy of a patient, wherein the first device is designed and arranged such to activate an operational mode of the first device in dependence on the data transferred by the second device. According to this aspect of the present disclosure, the second device is a drug pump, wherein the activated operational mode is a normal mode in which the first device is operated with standard functionalities or a pain patient mode in which an interference resistance of the first device is increased and/or in which the first device applies only a soft or assisting pacing since the patient is typically (extremely) exhausted by the pain treatment. An assisting pacing can be a pacing with an adapted timing of the individual stimulation pulses, resulting in a less harsh stimulation of the patient's heart.

In an embodiment of this aspect, the data switching the first device into the pain patient operational mode is sent by the drug pump only after having injected a drug bolus.

An independently claimed aspect of the present disclosure relates to an arrangement comprising a first device and a second device, wherein the first device and the second device can be operatively coupled with each other to transfer data at least from the second device to the first device, wherein the first device is an implanted medical device for cardiac therapy of a patient, wherein the first device is designed and arranged such to activate an operational mode of the first device in dependence on the data transferred by the second device. According to this aspect, the second device is a spinal-cord stimulation (SCS) device. In addition, the activated operational mode of the first device is a pain stress mode in which the stimulation rate is adapted to the pain stress expected for the patient due to the SCS. In addition, an interference resistance of the first device is increased in the pain stress mode with respect to the normal mode so that a reliable pacing is still possible even though the patient is in a state of increased pain stress. In such an arrangement, the first device, i.e., the medical device for cardiac therapy of the patient, can be included into a closed-loop stimulation control loop of the SCS device.

In an embodiment of this aspect, the first device provides the SCS device with the data on the cardiac rhythm of the patient. Then, the SCS device can adapt the stimulation applied to the spinal cord of the patient to the patient's cardiac rhythm. This will result in a more efficient pain treatment.

An aspect of the present invention relates to a method of operating a first device and a second device, in particular the first device and the second device of an arrangement according to the preceding explanations. In this context, data is transferred at least from the second device to the first device, wherein the first device is an implanted medical device for cardiac therapy of a patient, wherein an operational mode of the first device is activated in dependence on the data transferred by the second device.

According to this aspect, the second device is not a programming device serving for programming a medical device. In addition, the activated operational mode is chosen from the group consisting of a normal mode in which the first device is operated with standard functionalities; a protected mode in which the first device is operated without specific safety functions since a failure of the first device would be compensated by the second device; a danger mode in which the first device will deliver, during a first predeterminable period of time, cardiac pacing pulses with an increased amplitude and/or an increased rate; a magnetic resonance imaging mode in which the first device is deactivated (e.g., for a second predeterminable period of time) and subsequently reactivated after the second predeterminable period of time has passed or upon an absence of a previously present magnetic field; an increased stress mode being adapted to an expected increased level of stress of the patient; a fitness mode being adapted to an expected increased level of physical activity of the patient; a mobility mode in which a motion sensor of the first device is deactivated.

In an embodiment, the operational mode is activated upon receiving the data, which is transferred from the second device, by the first device.

All embodiments of the arrangement can be combined in any desired way and can be transferred either individually or in any arbitrary combination to the different individually claimed aspects and to the method. Furthermore, all features and embodiments of the different individually claimed aspects can be combined in any desired way and can be transferred either individually or in any arbitrary combination to the claimed arrangement, to any other individually claimed aspect, and to the claimed method. Likewise, all embodiments of the method can be combined in any desired way and can be transferred either individually or in any arbitrary combination to the claimed arrangement as well as to the different individually claimed aspects of the arrangement. Finally, the method for operating the arrangement cannot only be applied to the claimed arrangement, but also in an analogous way to the individually claimed aspects of the present disclosure.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWING

Further details of aspects of the present invention will be explained in the following making reference to an exemplary embodiment and an accompanying Figure. In the Figure:

FIG. 1 shows a schematic depiction of an embodiment of an arrangement.

### DETAILED DESCRIPTION

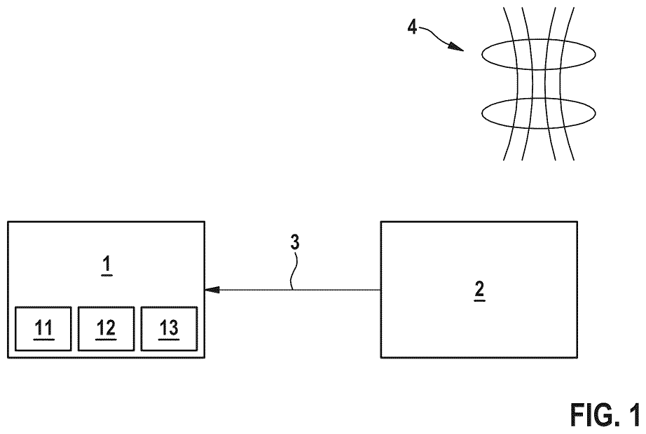

FIG. 1 schematically shows an implantable leadless pacemaker (iLP) 1 that serves as first device. The iLP 1 is operatively coupled with a subcutaneously implanted cardioverter-defibrillator (S-ICD) 2. The iLP 1 and the S-ICD 2 are implanted to the same patient. When this patient is subjected to a magnetic field 4, the S-ICD 2 will detect this magnetic field 4 earlier than the iLP 1 since it is located closer to the patient's skin and thus to a surface of the patient.

The S-ICD 2 will then send data 3 to the iLP 1, indicating that the patient is subject to the magnetic field 4. The S-ICD 2 will additionally determine whether the patient is in an upright or in a lying position and will include such information in the data 3 transferred to the iLP 1.

If the patient is an upright position, this will be taken as indication that the patient is currently at a security check, e.g., at an airport. The iLP 1 will then choose a magnetic resonance imaging mode 11 as operational mode to be activated. This magnetic resonance imaging mode 11 is one of a plurality of operational modes 11, 12, 13 that can be activated depending on the data 3 provided by the S-ICD 2.

Due to the additional information provided by the S-ICD 2 that the patient is an upright position, the iLP 1 will be operated in a variant of the magnetic resonance imaging mode 11 in which the iLP 1 is activated for, e.g., 2 minutes. After this time has passed, it is assumed that the patient has already left the magnetic field 4 since the security check is accomplished. Therefore, the iLP 1 can be automatically reactivated after these 2 minutes and operate again in its normal mode. If the S-ICD 2 again detects the presence of a magnetic field, the procedure can start from the beginning.

Due to the interaction between the S-ICD 2 and the iLP 1, the iLP 1 is able to indirectly “detect” the magnetic field 4 much earlier than without any assistance by the S-ICD 2. Therefore, it can be earlier deactivated when the patient is exposed to the magnetic field 4. This will result in a significantly reduced risk for damages or malfunctions of the iLP 1.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. An arrangement comprising a first device and a second device, wherein the first device and the second device can be operatively coupled with each other to transfer data at least from the second device to the first device, wherein the first device is an implanted medical device for cardiac therapy of a patient, wherein the first device is designed and arranged such to activate an operational mode of the first device in dependence on the data transferred by the second device,
wherein the second device is not a programming device serving for programming a medical device and in that the activated operational mode is chosen from the group consisting of:
a normal mode in which the first device is operated with standard functionalities and at least one second mode, wherein the second mode is chosen from at least one of:
a protected mode in which the first device is operated without specific safety functions since a failure of the first device would be compensated by the second device;
an increased stress mode being adapted to an expected increased level of stress of the patient, wherein the increased level of stress of the patient is detected based on an increase heart rate and/or an increased blood pressure, and wherein the increased stress mode comprises an adapted rate and/or an adapted timing of a pacing based on the increase heart rate and/or an increased blood pressure;
a fitness mode being adapted to an expected increased level of physical activity of the patient, wherein the fitness mode is determined by an increased heart rate and respiratory rate of the patient, and wherein the fitness mode comprises adjusting the pacing rate based on the increased heart rate and respiratory rate of the patient;
a mobility mode in which a motion sensor of the first device is deactivated.

2. The arrangement according to claim 1, wherein the first device is an implantable pulse generator, an implantable leadless pacemaker, or a subcutaneously implantable cardioverter-defibrillator.

3. The arrangement according to claim 1, wherein the second device is an implanted medical device for cardiac therapy of the patient, in particular an implantable pulse generator, an implantable leadless pacemaker, or a subcutaneously implantable cardioverter-defibrillator.

4. The arrangement according to claim 1, wherein the second device is a device providing location information of a user of the second device.

5. The arrangement according to claim 4, wherein the second device is a smartphone.

6. The arrangement according to claim 1, wherein the first device is an implantable pulse generator or an implantable leadless pacemaker and in that the second device is an implantable pulse generator or an implantable leadless pacemaker, wherein the data provided by the second device indicates that the second device has an essentially fully charged battery, wherein the activated operational mode of the first device is the protected mode.

7. The arrangement according to claim 1, wherein the first device is an implantable pulse generator or an implantable leadless pacemaker and in that the second device is a subcutaneously implantable cardioverter-defibrillator, wherein the data provided by the second device indicates that the second device has emitted a defibrillation pulse, wherein the activated operational mode of the first device is the danger mode.

8. The arrangement according to claim 7, wherein in addition to the modes for the second mode, a danger mode can be chosen, wherein in the danger mode, the first device will deliver, during a first predetermined period of time, cardiac pacing pulses with an increased amplitude and/or an increased rate,
Wherein the first device is designed and arranged such to switch the operational mode of the first device back from the danger mode to a normal mode after the first predeterminable period of time has passed.

9. The arrangement according to claim 1, wherein in addition to the modes for the second mode, a magnetic resonance imaging mode can be chosen, wherein in the magnetic resonance imaging mode, the first device is deactivated and subsequently reactivated after a second predeterminable period of time has passed or upon an absence of a previously present magnetic field,
and wherein the first device is an implantable pulse generator or an implantable leadless pacemaker and in that the second device is a subcutaneously implantable cardioverter-defibrillator, wherein the data provided by the second device indicates that the patient is subject to a magnetic field, wherein the activated operational mode of the first device is the magnetic resonance imaging mode.

10. The arrangement according to claim 9, wherein the first device is designed and arranged such i) to deactivate a pacing function of the first device if the patient is in a lying position and to reactivate the pacing function and to switch the operational mode of the first device to a normal mode after having been provided with data from the second device that the patient is no longer subject to the magnetic field; and/or ii) to deactivate the pacing function of the first device for the second predeterminable period of time if the patient is in an upright position and to reactivate the pacing function and to switch the operational mode of the first device to the normal mode after the second predeterminable period of time has passed.

11. The arrangement according to claim 10, wherein the first device and/or the second device comprise a position sensor for determining whether the patient is in a lying position or in an upright position.

12. The arrangement according to claim 4, wherein the activated operational mode of the first device is chosen in dependence on the location information, wherein the normal mode is activated if the location information indicates that the patient is at home, and/or wherein the increased stress mode is activated if the location information indicates that the patient is at work, and/or wherein the fitness mode is activated if the location information indicates that the patient is at a place at which the patient typically performs sportive activities, and/or wherein the mobility mode is activated if the location information indicates that the patient is in a moving car.

13. The arrangement according to claim 1, wherein the mobility mode additionally features the functionalities of the increased stress mode.

14. A method for operating a first device and a second device, in particular a first device and a second device of an arrangement according to claim 1, wherein data is transferred at least from the second device to the first device, wherein the first device is an implanted medical device for cardiac therapy of a patient, wherein an operational mode of the first device is activated in dependence on the data transferred by the second device,
wherein the second device is not a programming device serving for programming a medical device and in that the activated operational mode is chosen from the group consisting of:
a normal mode in which the first device is operated with standard functionalities and at least one second mode, wherein the second mode is chosen from at least one of:
a protected mode in which the first device is operated without specific safety functions since a failure of the first device would be compensated by the second device;
an increased stress mode being adapted to an expected increased level of stress of the patient, wherein the increased level of stress of the patient is detected based on an increase heart rate and/or an increased blood pressure, and wherein the increased stress mode comprises an adapted rate and/or an adapted timing of a pacing based on the increase heart rate and/or an increased blood pressure;
a fitness mode being adapted to an expected increased level of physical activity of the patient, wherein the fitness mode is determined by an increased heart rate and respiratory rate of the patient, and wherein the fitness mode comprises adjusting the pacing rate based on the increased heart rate and respiratory rate of the patient;
a mobility mode in which a motion sensor of the first device is deactivated.

15. The method according to claim 14, wherein the operational mode is activated upon the data transferred by the second device is received by the first device.
